(19) United States
(12) Patent Application Publication
(10) Pub. No.: US 2022/0047745 A1
(43) Pub. Date: Feb. 17, 2022

(54) METHODS FOR DETECTING A MEDICAL INSTRUMENT IN A DISINFECTION SYSTEM AND DISINFECTION SYSTEMS IMPLEMENTING THE METHODS

(71) Applicant: GERMITEC, IVRY-SUR-SEINE (FR)

(72) Inventors: Cédric NEVEU, Montrouge (FR); Frédéric LEPINE, Saint-Gratien (FR); Clément DESHAYS, Paris (FR)

(21) Appl. No.: 17/434,602

(22) PCT Filed: Feb. 26, 2020
(86) PCT No.: PCT/EP2020/054989

§ 371 Date: Aug. 27, 2021

(30) Foreign Application Priority Data
Feb. 27, 2019 (FR) 1902021

(51) Int. Cl. A61L 2/10; A61L 2/26

(52) U.S. Cl. CPC A61L 2/10; A61L 2/26; A61L 2202/24; A61L 2202/14; A61L 2202/122

(57) ABSTRACT

The present description relates to a disinfection system (300) configured to implement a disinfection cycle of a medical instrument (200) by UV radiation. The disinfection system (300) includes a disinfection chamber (310) configured to receive at least one portion of the medical instrument (200), a suspension means (320) for suspending said at least one portion of the medical instrument (200) in said disinfection chamber (310), a detection device (330) configured to detect, in a predetermined detection area of the disinfection system (300), a marker (210) integral with said medical instrument (200), and a controller (370) configured to generate a non-detection signal in case of non-detection of said marker (210) in said predetermined detection area.

### TECHNICAL SCOPE OF THE INVENTION

The present description relates generally to methods for detecting a medical instrument in a disinfection system, and more specifically a UV radiation disinfection system. The present description also relates to disinfection systems implementing such methods of detecting a medical instrument as well as disinfection kits for medical instruments.

### STATE OF THE ART

Proper disinfection or sterilization of reusable medical instruments is important to prevent the transmission of pathogenic microbes from one person to another. The level of sterilization and disinfection applied to medical devices depends on the classification of the device.

Classes of critical instruments that come into contact with sterile tissue area specifically distinguished, such as surgical instruments, implants and ultrasound probes used in sterile body cavities. These instruments must be sterilized or undergo High-Level Disinfection (HLD) as a minimum before use. Classes of semi-critical instruments that typically come into contact with mucous membranes or non-intact skin are distinguished. Examples of semi-critical instruments include devices such as probes used in vaginal, rectal and urological examinations, respiratory therapy and anesthesia equipment, and certain endoscopes. These medical devices must be free of all microorganisms, except for a small number of spores. Semi-critical devices require at least Intermediate-Level Disinfection (ILD). Non-critical instruments are those that come in contact with non-mucosal membranes of intact skin (e.g., blood pressure cuffs and stethoscopes).

Some common methods for achieving sterilization or High-Level Disinfection of critical or semi-critical instruments include treatments using steam and/or chemical disinfectants (see, for example, the Nanosonics® trophon® chemical disinfectant system).

Systems capable of achieving High-Level Disinfection of reusable medical instruments by means of ultraviolet (UV) radiation are also known. Compared to treatments using steam and/or chemical disinfectants as described above, these techniques allow for rapid High-Level Disinfection at a low temperature, without potentially toxic products for the personnel handling them and in clinical conditions of use.

For example, patent application US2014341777, published in the name of the applicant, describes a disinfection system using a disinfection chamber with a UV radiation source.

FIG. 1 describes a disinfection chamber 110 of a disinfection system as disclosed in the aforementioned application US2014341777. The disinfection chamber generally comprises an enclosure 102 with a plurality of side walls 104, a ceiling 106, a door 108 arranged on one of the side walls 104, for example, to access an interior volume 112 of the disinfection chamber through an opening 109 formed in one of the walls. In the example of FIG. 1, a suspension assembly 114 is provided for suspending a medical instrument to be disinfected, during a disinfection cycle, in a central region of the interior volume of the chamber 110 in which high intensity UV radiation is generated. Thus, for example, when the medical instrument to be disinfected (not shown in FIG. 1) comprises a head (or active portion) and a connection cable, the suspension assembly may comprise a slot 116 opening into the opening 109 and at one end of which there is an opening 122 through which the cable of a medical instrument may pass. For example, the opening 122 may interact with a ring attached to the connection cable at a position determined for the head to be in the disinfection chamber, in a position that allows for optimal disinfection.

In other exemplary embodiments (not shown), the suspension assembly may comprise a clamp configured to clamp the connection cable at a determined position.

However, regardless of how the medical instrument is positioned in the disinfection chamber, the applicant has identified certain drifts in the use of disinfection systems resulting from improper positioning of the medical device. As illustrated in FIGS. 2A through 2D, the misplacement may result from axial slippage of the connection cable in the suspension assembly, for example, this slippage capable of resulting in uncontrolled displacement of the position of the head in the chamber, or from a manual error in hanging the instrument.

By diagrams, FIGS. 2A through 2D thus illustrate situations in which a medical instrument 200, formed by a head 201 and a connection cable 202, is suspended at different positions in the interior volume 112 of a UV radiation disinfection chamber 110.

In the example of FIG. 2A, the medical instrument 200 is suspended such that the head 201 is substantially positioned in a central region of the interior volume of the chamber 110, such as a predetermined position for optimal disinfection. The predetermined position for optimal disinfection is a position to deliver a sufficient UV dose to the least exposed point of the instrument head, for example, to achieve high-level disinfection there, and thereby ensure high-level disinfection over the entire surface of the instrument head. In the example of FIG. 2B the connection cable 202 is not or is no longer attached by the same point in the suspension assembly, for some reason, so that the head 201 is near the ceiling. The region of the head 201 indicated by arrow A can no longer receive the expected optimal UV radiation amount needed to disinfect it. In the example shown in FIG. 2C, the head 201 is near the bottom of the chamber 110. Again, the region of the head 201 indicated by arrow B can no longer receive the expected optimal UV radiation amount needed to disinfect it. In this same position of the medical instrument 200 toward the bottom of the chamber, as illustrated in FIG. 2D, certain regions of the head 201 indicated by arrow C could instead receive a much greater UV radiation amount specifically due to the position of the UV sources, for example, risking damage to the head 201 of the instrument.

Thus, an uncontrolled position of the medical instrument during a disinfection cycle can result in uncontrolled, sometimes insufficient disinfection of the medical instrument. Undetected, this poor position may thus result in a medical instrument being reused after disinfection under unsatisfactory sanitary conditions.

An object of the present description is to remedy the aforementioned disadvantages specifically by means of original disinfection systems and methods for detecting a medical instrument in such disinfection systems, said methods aiming to verify that the medical instrument is in a predetermined position during a disinfection cycle.

### SUMMARY OF THE INVENTION

According to a first aspect, the present description relates to a disinfection system configured to implement a disinfection cycle of a medical instrument by UV radiation. The disinfection system comprises a disinfection chamber configured to receive at least one portion of said medical instrument, a suspension means for suspending said at least one portion of the medical instrument in said disinfection chamber, a detection device configured to detect a marker in a predetermined detection area of the disinfection system, integral with said medical instrument, and a controller configured to generate a non-detection signal in case of non-detection of said marker in said predetermined detection area.

An “integral marker” means a fixed marker that cannot be removed and/or moved without damaging said marker and/or the medical instrument to which it is affixed. Thus, the marker can be an external element (such as a label, an electronic chip) affixed in an integral manner to the medical instrument by gluing or integration within the medical instrument, for example, or an element of the medical instrument itself.

A disinfection system designed in this way can be used to warn of an anomaly in the positioning of the medical instrument and therefore possibly of a risk of non-optimal disinfection. Indeed, the fact that the marker is integral with the medical instrument makes it possible to control its position in the disinfection system with certainty, regardless of the type of disinfection means.

According to one or more embodiments, said marker detection area is determined to ensure that said at least one portion of the medical instrument receives a minimum UV dose on its least exposed portion, such as a dose enabling high-level disinfection. Detection of the marker therefore ensures high-level disinfection over the entire portion of the instrument intended to be received in the disinfection chamber.

According to one or more exemplary embodiments, the detection device comprises an optical detection device. However, other types of detection device are possible. Thus, according to one or more exemplary embodiments, the detection device may comprise means of acquisition by radio frequency.

According to one or more exemplary embodiments, the controller is configured to generate one or more actions from the non-detection signal, chosen from among: issuing an alert to one or more users, prohibiting the launching of a disinfection cycle, a stop (blocking) of a disinfection cycle in progress.

According to one or more exemplary embodiments, the detection device, whether it is an optical or radio frequency detection device, may be further configured to detect a marker's position relative to a reference position in said detection area.

According to one or more exemplary embodiments, the controller is configured to generate one or more actions from said marker's position in the detection area, comprising: emitting a positioning error alert to one or more user(s) and/or reconfiguration of a disinfection cycle to take account of said position and/or stopping the disinfection cycle in progress and, optionally, switching to error mode, and/or prohibiting the launching of a disinfection cycle.

According to one or more exemplary embodiments, the detection device is an optical detection device, i.e., a detector sensitive to visible or invisible radiation, such as infrared radiation, for example.

An optical detection device of a disinfection system according to the present description may comprise, for example and in a non-limiting manner, a detector selected from the group comprising: a point detector, e.g., a photodiode, a matrix detector, e.g., a line camera or a two-dimensional camera, e.g., a CCD or CMOS camera, e.g., a one- or two-dimensional barcode reader.

Of course, the detection device is adapted according to the marker. For example, a camera is adapted to a marker formed by a structural element of the medical instrument itself and identified as a marker for the device. For example, a point detector such as a photodiode is adapted to a point marker, for example in the form of a red dot brought to the cable, while a barcode reader is adapted to a barcode type marker.

According to one or more exemplary embodiments, the detection device is an optical detection device and further comprises a source of light radiation emission configured to illuminate said predetermined detection area. The emission source enables detection even when the detection area is in a non-illuminated region of the disinfection system, during an operating cycle, such as a space closed to daylight.

According to one or more exemplary embodiments, the detection device is configured to detect a marker located in said portion suspended in the disinfection chamber during a disinfection cycle and/or in a portion of the medical instrument that remains outside the disinfection chamber during a disinfection cycle.

The disinfection system according to the first aspect is configured to implement a UV radiation disinfection cycle. The disinfection system may then comprise any means known in the state of the art for implementing a UV radiation disinfection cycle, and specifically at least one UV radiation source.

According to a second aspect, the present description relates to a detection kit for a medical instrument to be disinfected in a disinfection system according to the first aspect. The detection kit includes a marker configured to be integrally affixed to said medical instrument for detection by said detection device.

A detection kit according to the second aspect may be used by an operator upstream of a disinfection cycle to “mark” a medical instrument for detection in the disinfection system before and/or during and/or after a disinfection cycle.

The marker may comprise any element that can be specifically detected by the detection device.

For example, when the detection device is an optical detection device, the marker may include any element that is “visible” to the detector, optionally when illuminated by an emission source of the detection device. According to one or more examples and in a non-limiting manner, the marker may comprise an element selected from the group consisting of: a colored element, a fluorescent element, a screen print, a reflective element, a diffractive element, a bar code, or a combination of these elements.

According to one or more exemplary embodiments, the marker comprises a code for identifying said medical device. For example, the marker comprises a one- or even two-dimensional barcode. Such a marker may be detected by an optical detection device such as a barcode reader. An identification of the device may include a serial number and/or a model number, according to one or more exemplary embodiments. Thus, the reading of the identification of the medical instrument can be coupled with detection thereof in the disinfection system.

According to one or more exemplary embodiments, when the medical instrument comprises a head and a cable connecting the head, the marker may be configured to be integrally affixed to a portion of the cable adjacent to the head. This may be a portion of the cable intended to be suspended in the disinfection chamber during a disinfection cycle or a portion of the cable intended to remain outside the disinfection chamber.

According to one or more exemplary embodiments, the marker is an element configured to be integrally affixed to said medical instrument by gluing, molding or welding.

According to one or more exemplary embodiments, the detection kit further comprises a protective ring for said marker, said protective ring further interacting with said suspension means for suspension of the cable.

For example, when the marker comprises an element allowing optical detection by the detection device, said protective ring may comprise a window transparent to the wavelengths used for detection. Such a protective ring, available as a flexible label for example, will protect the marker during a disinfection cycle.

According to a third aspect, the present description relates to a disinfection kit for a medical instrument comprising a disinfection system according to the first aspect and a detection kit according to the second aspect.

According to a fourth aspect, the present description relates to a marked medical instrument, intended to be disinfected in a disinfection system according to the first aspect. Said marked medical instrument includes a marker integrally affixed to a region of said medical instrument for detection by said detection device. The marker is thus formed of an element external to the instrument and configured for detection of the medical instrument when positioned in the disinfection system, for a disinfection cycle. According to one or more exemplary embodiments, said marker comprises an identification code for the medical instrument. Such a marked medical instrument may be provided with the disinfection system, for example.

According to a fifth aspect, the present description relates to a method for detecting a medical instrument in a disinfection system according to the first aspect.

A method for detecting a medical instrument according to the present description comprises the following steps:
detecting said marker integral with said medical instrument in said predetermined detection area of the disinfection system, by means of said detection device; and
in case of non-detection of said marker in said predetermined detection area of the disinfection system, generating a non-detection signal.

Detection of the marker may occur before and/or during and/or at the end of a disinfection cycle.

According to one or more exemplary embodiments, the generating of the non-detection signal results in one or more actions, including: a prohibition of the launching of a cycle and/or emission of a cycle invalidation alert to a user and/or a stop (blocking) of a disinfection cycle in progress.

According to one or more exemplary embodiments, the detection is an optical detection.

According to one or more exemplary embodiments, the marker comprises an identification code of the medical instrument and the method further comprises reading the identification code using the detection device.

According to one or more exemplary embodiments, the detection method further comprises detecting a marker position relative to a reference position, in said detection area.

According to one or more exemplary embodiments, the detection method also comprises one or more actions, from said marker position in the detection area, comprising: issuing a positioning error alert to one or more users and/or reconfiguring a disinfection cycle to take account of said position, and/or stopping the disinfection cycle in progress and, optionally, switching to error mode, and/or a prohibition of the launching of a disinfection cycle.

According to one or more exemplary embodiments, the method further comprises a prior step of marking the medical instrument, by affixing said marker integrally to said medical instrument.

### BRIEF DESCRIPTION OF THE FIGURES

Further advantages and features of the invention will become apparent from the description, illustrated by the following Figures:

FIG. 1 (previously described), a diagram of a disinfection enclosure according to the state of the art;

FIGS. 2A through 2D (previously described), diagrams illustrating positions of a medical instrument including a head and a cable for connecting the head in a disinfection system according to the state of the art, in cases where the head is positioned in a central and optimized region of the chamber (FIG. 2A), in cases where the head is positioned in a region of the chamber that is too high (FIG. 2B), and in cases where the head is positioned in a region of the chamber that is too low (FIGS. 2C, 2D);

FIGS. 3A, 3B, diagrams illustrating an example of a disinfection system according to the present description, with a medical instrument suspended and detected by the detection device in a first case (FIG. 3A) and a medical instrument suspended and not detected by the detection device in a second case (FIG. 3B);

FIGS. 4A, 4B, diagrams illustrating a protective ring of a medical instrument detection kit according to the present description;

FIGS. 5A, 5B, diagrams illustrating an example support of a disinfection system suspension means according to the present description, intended to interact with a protective ring as illustrated in FIGS. 4A, 4B;

FIG. 6, a diagram diagram illustrating another exemplary embodiment of a disinfection system according to the present description;

FIGS. 7A, 7B, 7C, 7D, diagrams illustrating examples of implementation of methods for detecting a medical instrument in a disinfection system, according to the present description.

### DETAILED DESCRIPTION

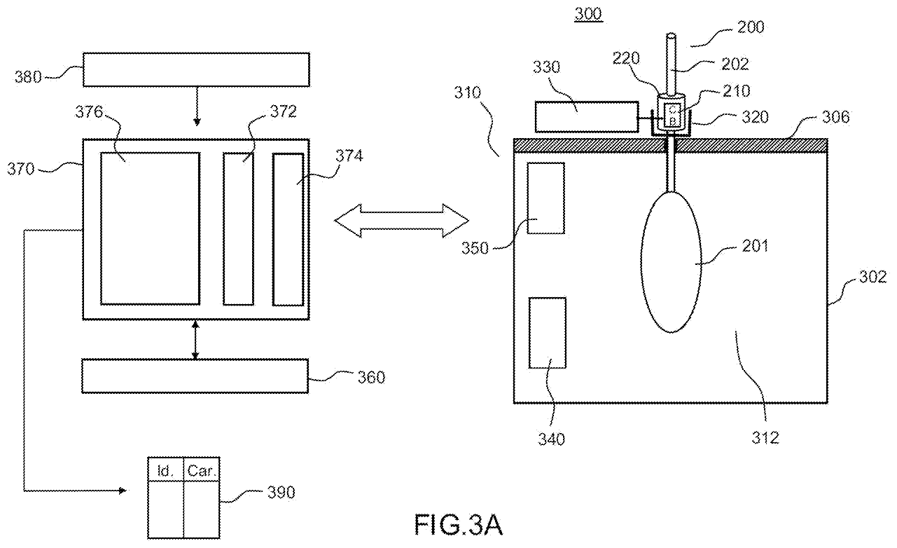

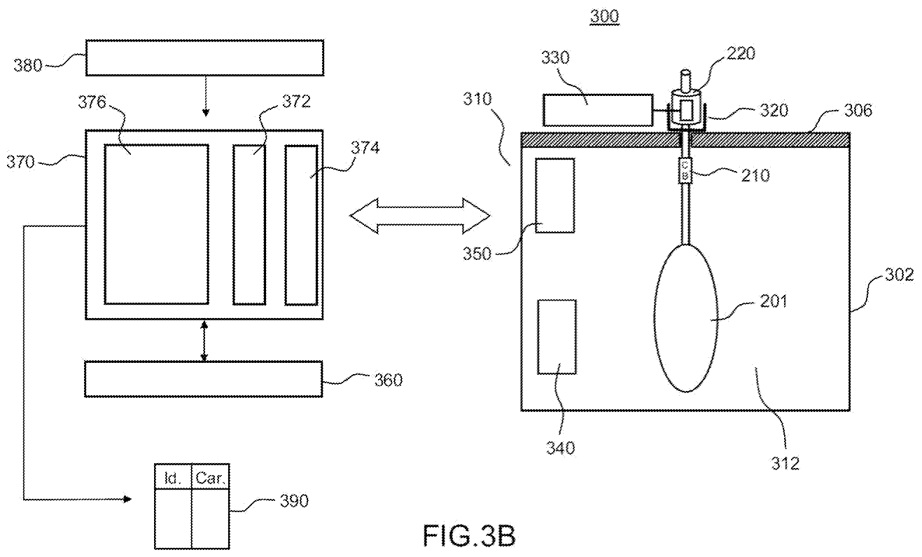

FIG. 3A schematically depicts an example of a disinfection system 300 according to the present description, in a cross-sectional view.

The disinfection system 300 is configured to implement a disinfection cycle of at least one medical instrument, designated by reference 200 in FIG. 3A.

Although the present description is not limited to disinfection methods or systems for disinfecting critical and semi-critical medical instruments, the methods and systems described in the present description are specifically suited for HLD or ILD of reusable medical devices and instruments, including, for example, ultrasound, endotracheal, and other endo-cavity probes such as ENT endoscopes.

Such a medical instrument comprises an active portion or “head” 201, for example, and a head connection cable, designated 202, although medical instruments without connection cables may also be disinfected using the disinfection methods and systems described in this description.

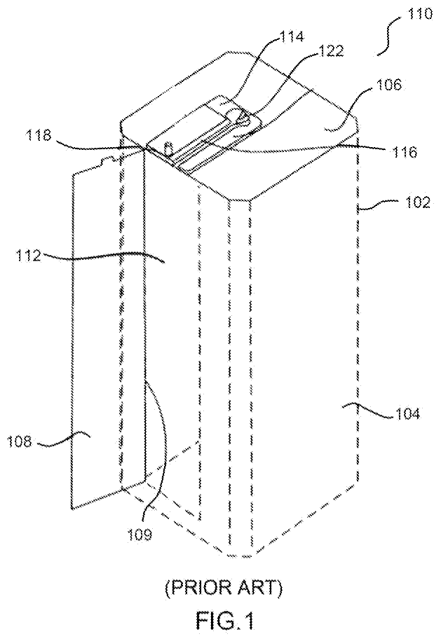

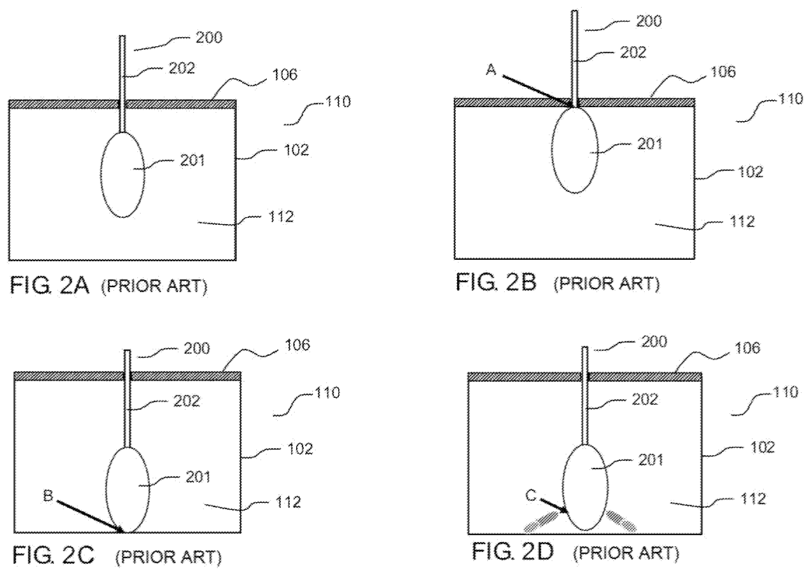

In the example of FIG. 3A, the disinfection system 300 comprises one or more disinfection chamber(s) 310 with an enclosure 302 of the type shown in FIG. 1, for example. The disinfection chamber 310 comprises side walls and a ceiling 306. The disinfection chamber 310 is configured to receive at least one portion of the medical device within an interior volume 312, such as the head 201, and one portion of the connection cable 202.

In the present description, the methods and systems described in the present description use ultraviolet (“UV”) radiation to rapidly perform high-level disinfection without generating excessively high temperatures on the surface of the instruments to be treated and inside them.

Thus, in the example of FIG. 3A, the disinfection system is configured to implement a UV radiation disinfection cycle.

In a manner known in the state of the art, it comprises one or more UV radiation sources, referenced generally by reference 350, and one or more sensors of said UV radiation, referenced generally by reference 350. The disinfection system may include other sensors in a manner known in the state of the art, such as temperature sensors.

Radiation sources that may be used in methods and systems according to the present description are sources known in the state of the art, for example, and include radiation sources for emitting UV-A, UV-B or UV-C radiation, for example.

UV-C radiation sources, also referred to as “lamps” or “tubes”, are commercially available and can be obtained in various shapes, sizes, radiation and output energy. Examples of UV-C tubes suitable for use as UV-C radiation sources include low pressure mercury vapor discharge lamps. However, in the methods and systems according to the present description, any source capable of emitting UV-C radiation in the selected UV-C wavelength at an output energy that contributes to the disinfection of a target medical instrument 200 could be used. For example, in addition to or instead of one or more UV-C tubes, one or more lasers or photodiodes, or arrays of sources, or combinations of types of sources designed to emit UV-C light may be used to deliver radiation into the disinfection chamber.

The source(s) 350 may also comprise one or more indirect sources of UV radiation (e.g., dedicated radiation reflectors).

The one or more UV radiation sensors 340, such as one or more photodiodes, may be fixedly or movably positioned within the interior volume of the disinfection chamber 310, and are adapted to determine an amount of UV radiation, for example a radiation amount measured in joules on a selected surface or, in some cases, a surface-specific radiation amount for example, measured in joules/cm². The one or more UV radiation sensors arranged within the interior volume of the disinfection chamber 310 may be configured to have a bandpass optical filter or other electromagnetic filter so that only radiation in a spectrum of interest is detected.

In other embodiments, the sensor(s) 340 may comprise one or more light conducting components such as lenses, optical fibers, mirrors, filters, and/or other optical elements used to collect radiation in the interior volume of the chamber 310 to a detector, such as a photodiode.

The disinfection chamber 310 is sized and configured to help achieve disinfection of instruments 200 placed therein within a desirable, possibly selectable period of time such that the surfaces of the instruments are exposed to a desired radiation level, referred to as “dosage” in this description. A radiation exposure level, or dosage, refers to both the intensity of the radiation and the duration of the exposure. For example, the instrument to be disinfected, the UV radiation source(s), and/or the UV radiation sensor(s) are positioned (e.g., inserted, interposed, suspended, or located) in the disinfection chamber at fixed or non-stationary positions, the positions being able to be non-stationary during a disinfection cycle, to better expose each of the surface portions of the instrument to specific, pre-determined disinfection levels of UV radiation.

The disinfection system further comprises a detection device 330 configured to detect a marker 210 in a predetermined detection area of the disinfection system, integral with a medical instrument 200 that is to undergo a disinfection cycle. Examples of markers 210 and detection device 330 will be described in more detail later.

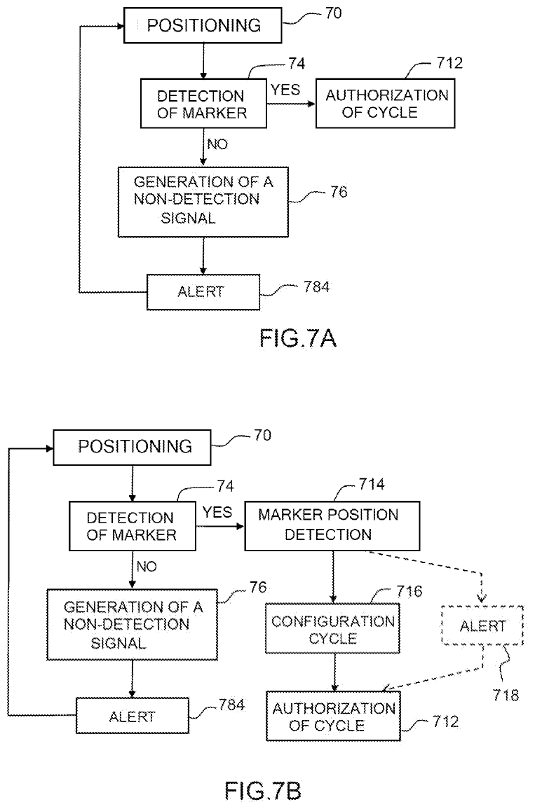

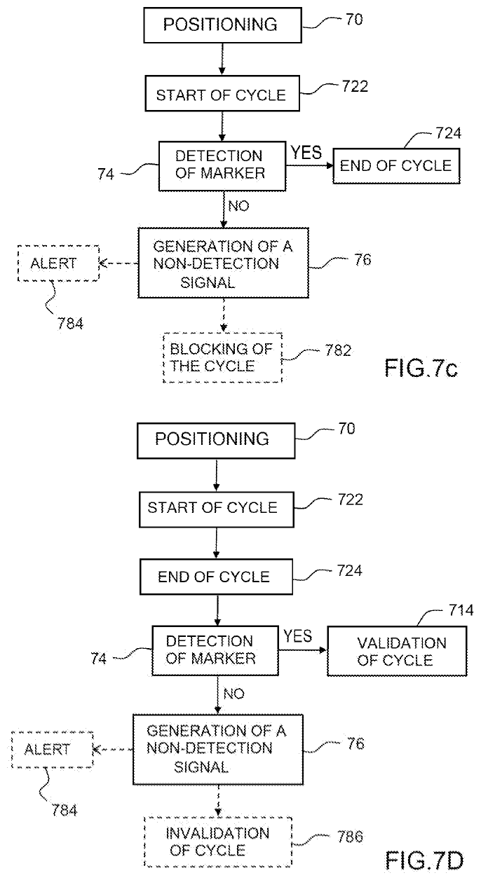

As will be described in more detail by means of FIGS. 7A - 7D, a controller 370 is configured to generate a non-detection signal, before and/or during and/or after a disinfection cycle, in the event of non-detection of the marker 210 in the predetermined detection area, and generate one or more actions, if necessary.

Generally, the disinfection chamber 310 and/or radiation source(s) 350 and/or UV radiation sensor(s) 340 and/or detection device 330 and/or other sensors arranged in the disinfection chamber are coupled in communication with the controller 370. In addition to exchanging data with the disinfection chamber, radiation sources, sensors, and marker detection device, the controller 370 may also communicate with one or more databases 360 and/or an input interface 380 for data/information related to the control of the disinfection operation, to perform its operations.

The information relating to the control of the disinfection operation may include information characterizing the decontamination cycle, such as information identifying the probe and/or the enclosure, each enclosure then being assigned a specific identification number stored therein, time-stamping information of the cycle enabling, for example, the acquisition of the date of the cycle, the daily number of the cycle, the start time and the end time of the cycle, from a clock-forming circuit, etc. The decontamination cycle characterization information may also include information relating to the UV dose emitted during a cycle if the enclosure is a disinfection enclosure provided with means of generating disinfection UV radiation. For example, in order to ensure the traceability of the cycle, the information relating to the control of the disinfection operation may include information identifying the probe and/or the enclosure, information identifying the patient who will benefit from the disinfection, as well as information identifying the operator who performed the disinfection.

In this way, the controller can control the execution of a disinfection cycle, determine a minimum dosage to be applied in the disinfection chamber, for optimal disinfection of the instrument, and control the positioning of the instrument.

The controller 370 may comprises a processor 372, as known in the state of the art, one or more storage units or memories 376, and an interface 374 configured to communicate with the disinfection chamber 310, the radiation source(s) 350, the sensor(s) 340, the detection device 330. The interface 374 includes a CAN converter, for example, for converting analog data such as data detected by the sensors into digital data that can be processed by the processor 372. A processor 372 as described in this description includes any device such as a central processing unit (CPU), microprocessor, microcontroller (MCU), digital signal processor (DSP), application specific integrated circuit (ASIC), or portion thereof, that controls at least one operation; it may be implemented as hardware, firmware, or software, or a combination. The functionality associated with a specific processor may be centralized or distributed, either locally or remotely. A processor may refer interchangeably to any type of electronic control circuit configured to execute programmed software instructions. The one or more memories 376 may comprise any combination of volatile and non-volatile computer-readable media for reading and writing, as known in the state of the art. Volatile computer-readable media include random access memory (RAM), for example. Non-volatile computer-readable media includes one or more ROMs, magnetic media such as a hard disk, optical disk, flash memory device, CD-ROM, etc., for example, Some or all stored contents of a memory may include software instructions executable by a processing device to perform one or more specific acts.

Among the stored instructions, indicatively but without limitation, these instructions may include a plurality of accepted probes and a plurality of doses to be applied.

The marker 210 is integral with the medical device, i.e., it cannot be removed and/or moved without damage to the marker itself and/or the medical instrument to which it is affixed. The marker may be an external element affixed integrally to the instrument, by bonding or integration within the instrument for example, such as a label or an electronic chip. The marker can also be a portion of the instrument itself. With a marker integral with the instrument, the position of the medical instrument in the disinfection system can be monitored reliably.

The marker 210 may be carried by the active portion 201 of the instrument or alternatively by the connection cable 202 of the instrument, as shown in FIG. 3A.

The detection device 330 may be positioned outside the interior volume of the disinfection chamber, as illustrated in FIG. 3A, or within the interior volume 312, at the side walls thereof for example, if the marker is positioned on or near the active portion of the instrument for example.

Generally, the marker may comprise any element that can be specifically detected by the detection device.

For example, the marker may be an electronic chip of the RFID (“radio frequency identification”) type and in this case the detection device may comprise radio frequency acquisition means configured for the detection of the electronic chip.

According to other examples, the detection device 330 is an optical detection device, sensitive to visible or infrared radiation.

It may comprise one or more point detector(s), for example, such as one or more photodiode(s) and/or one or more array detector(s), such as a line camera or a two-dimensional camera, such as a CCD or CMOS camera. The detection device 330 may also include a one- or two-dimensional barcode reader for identifying the medical device, as will be described in more detail later.

Generally, the detection device is adapted according to the marker.

According to one or more examples and in a non-limiting manner, the marker may comprise an element selected from the group comprising: a colored element, a fluorescent element, a silk-screen print, a reflective element, a diffractive element, a barcode, or a combination of these elements.

For example, one or more cameras are adapted to a marker formed from a structural element of the medical instrument itself and identified as a marker for the instrument. A point detector, such as a photodiode, is adapted to a point marker such as a wave absorbing or reflecting point in a different proportion than the rest of the probe elements, while a barcode reader is adapted to a barcode marker. Another non-limiting example of optical detection may include a laser.

According to one or more exemplary embodiments, the detection device further comprises one or more light emission source(s) configured to illuminate said detection area. The emission source enables detection even when the detection area is in a non-illuminated region of the disinfection system, during an operating cycle, such as a space closed to daylight.

According to one or more exemplary embodiments, the detection device is further configured to detect a position of the marker relative to a reference position, in said detection area. In this case, the controller 370 may be configured to generate the reconfiguration of a disinfection cycle, based on said position of the marker in the detection area, to take into account said position.

According to an example of the present description, the marker 210 integral with the medical instrument comprises an identification code for the instrument. This identification code may be stored as a one- or two-dimensional bar code or in an electronic radio frequency tag, for example.

The detection device 330 and the controller 370 are then configured to acquire the identification information carried by the instrument identification code, in addition to detecting whether the instrument is present in the detection area. For example, the detection device 330 comprises a barcode reader in the case of a barcode type marker or radio frequency acquisition means in the case of a radio frequency electronic tag. The identification information of the medical instrument can be acquired when the medical instrument is placed in the disinfection chamber and/or when it is removed from the disinfection chamber and/or at the beginning and/or end of a disinfection cycle.

The information identifying the or each instrument, coupled with information detecting the instrument and characterizing the disinfection cycle, enables the generation of traceability information for the disinfection of the or each instrument referenced 390 in FIG. 3A. This traceability information makes it possible to relate each instrument to the conditions under which the corresponding disinfection cycle took place, for example, and may optionally be associated with information relating to the identity of the patient who benefited from the disinfection, and/or with information relating to the operator who performed the disinfection. This traceability information can be stored, visualized on a display or transmitted to one or more remote operators. It enables the passage of the instrument in the disinfection chamber to be guaranteed and the information characterizing the disinfection cycle undergone by the instrument to be checked, i.e. the specific moment when this disinfection took place, the chamber in which the disinfection cycle took place, the validation of good positioning of the instrument in the disinfection chamber, the UV dose received by the instrument, the temperature during the cycle, the fact that the instrument was well cleaned before the disinfection cycle.

According to one or more exemplary embodiments, the marker may be provided in the form of a detection kit for a medical instrument to be disinfected in a disinfection system according to the present description. The detection kit thus includes a marker configured to be integrally affixed to said medical instrument for detection by the detection device.

The detection kit may be used by an operator upstream of a disinfection cycle to “mark” a medical instrument for detection in the disinfection system before and/or during and/or after a disinfection cycle.

Advantageously, the detection kit marker can include an identification code of the medical device.

In practice, an operator may affix the marker integrally to the medical instrument at a predetermined position thereof. For example, an installer of the disinfection system could apply a barcode-type label at a certain point on the instrument's cable, cover this label with an element that encloses the cable and makes the label non-removable under normal use conditions. This element may be configured to allow for systematically repeatable attachment. According to one or more exemplary embodiments, the element may form a male-female pair with one of the portions of the enclosure, and may be provided with a transparent portion, for example a glass pane, which allows the rays of the identification code reader to pass through, for example, the rays of the bar code reader.

According to one or more exemplary embodiments, an RFID chip may be applied to a predetermined cable location and coupled to an RFID reader with a straight and restricted detection field. This embodiment may perform the function of detecting a marker position relative to a reference position within the detection area.

According to other exemplary embodiments, marked medical instruments configured to be disinfected in a disinfection system according to the present description may also be designed. Each marked medical instrument includes a marker integrally affixed to a region of said medical instrument for detection by the detection device. The marker is thus formed of an element external to the instrument and configured for detection of the medical instrument when positioned in the disinfection system, for a disinfection cycle. According to one or more exemplary embodiments, the marker comprises an identification code for the medical instrument. Such a marked medical instrument may be provided with the disinfection system, for example.

According to other exemplary embodiments, a marker may be integrated into already manufactured medical instruments. For example, according to one or more exemplary embodiments, a probe may incorporate an identifier in a cable, in a shape that may fit into a portion of interest of the disinfection chamber enclosure, positioning the identifier within the reader's detection field.

In the example of FIG. 3A, the marker 210 comprises a label with an identification code, for example; it is carried by the connection cable and is detected, in operation, by the detection device 330 such as a barcode reader arranged outside the interior volume of the disinfection chamber. The barcode reader presents a given field that defines a detection area.

As illustrated in FIG. 3B, if for some reason the medical instrument is not in the predefined position for disinfection, the optical sensor 330 does not detect in the detection area the marker, causing the controller 370 to generate a non-detection signal.

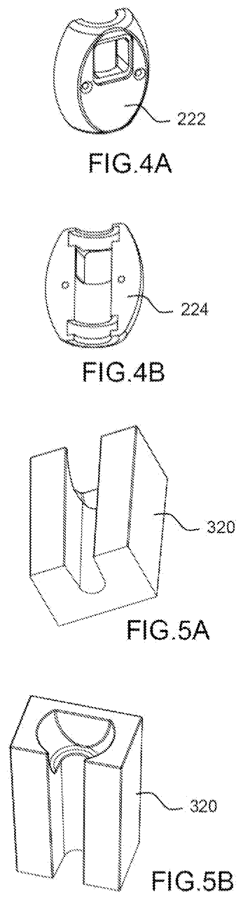

In the example of FIG. 3A, the disinfection chamber 310 is configured to allow the medical instrument to be suspended in the interior volume 312, by means of a ring 220 interacting with a suspension means 320 for example, as further described by means of FIGS. 4A, 4B and 5A, 5B.

The suspension means 320 shown in two views in FIGS. 5A and 5B comprises a body in which a cavity of complementary shape to at least a portion of the medical instrument is defined.

The suspension means interacts with the ring 220 configured to be attached to the cable 202 of the medical instrument 200, for example, so as to leave the marker 210 exposed. For example, in the case of optical detection of the marker 210, the ring 220 comprises a window 221 intended to be placed at the level of the marker. If the ring 220 slides unintentionally on the cable, as shown for example in FIG. 3B, the marker 210 integral with the medical instrument 200 will no longer be detected by the detection device 330.

Of course, other means may be contemplated for positioning the medical instrument in the chamber 310.

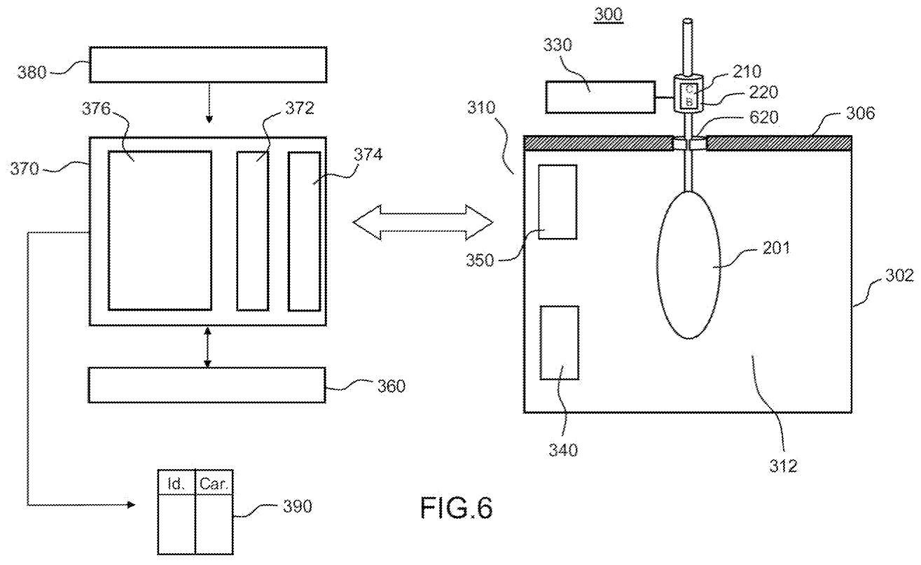

Thus, in the exemplary embodiment of a disinfection system in FIG. 6, a ring-shaped suspension means 620 housed in a ceiling opening 306 is illustrated.

FIGS. 7A through 7D depict diagrams illustrating examples of implementation of methods for detecting a medical instrument in a disinfection system according to the present description.

A method for detecting a medical instrument according to the present description generally comprises the detection 74 of a marker integral with said medical instrument in a predetermined detection area of the disinfection system, by means of a detection device; and, in case of non-detection of said marker in said predetermined detection area of the disinfection system, the generation 76 of a non-detection signal.

Detection of the marker may occur before and/or during and/or at the end of a disinfection cycle.

FIGS. 7A and 7B illustrate examples of methods wherein marker detection is done prior to initiation of a detection cycle. FIG. 7C illustrates an example of a method in which detection of the marker is done during a detection cycle and FIG. 7D illustrates an example of a method in which detection of the marker is done after a detection cycle. Of course, these examples are not limiting and may be combined.

Specifically, the example method illustrated in FIG. 7A comprises the detection 74 of the marker integral with the medical device, in the detection area, after positioning 70 of the medical device. If the marker is detected, the cycle may be enabled (712). If the marker is not detected in the detection area, the method includes generating 76 a non-detection signal. This may result in the generation of an alert (784) to a user who may check and correct the position of the medical instrument in the disinfection system.

The example method illustrated in FIG. 7B represents a variant of that illustrated in FIG. 7A in which, in the event of detection of the marker in the detection area, there is also a detection 714, in said detection area, of a position of the marker relative to a reference position. Based on said position of the marker in the detection area, one or more actions follow, including, for example: authorizing the launch of the cycle (712), issuing 718 a positioning error alert to one or more users and/or reconfiguring 716 a disinfection cycle to take account of said position.

The example method illustrated in FIG. 7C includes, after positioning 70 the medical instrument and initiating a cycle (722), detecting 74 within the detection area the marker integral with the medical device. If the marker is detected, the cycle can continue normally (724). If the marker is not detected in the detection area, the method comprises the generation 76 of a non-detection signal. This may result in the generation of an alert (784) to a user, for example, or the blocking of the cycle (782).

The example method shown in FIG. 7D comprises, after positioning 70 the medical instrument and completing a disinfection cycle (722, 724), detecting 74 in the detection area the marker integral with the medical instrument. If the marker is detected, the cycle is validated (714). If the marker is not detected in the detection area, the method comprises the generation 76 of a non-detection signal. This may result in the generation of an alert (784) to a user, for example, or an invalidation of the cycle (786).

In the example methods described above, the detection of the marker may occur according to any of the examples described above. For example, it may involve an optical detection of a marker that may take a variety of forms, including an external element (e.g., a label) for example, integrally affixed to the instrument, such as by bonding or integration within the instrument, or an element of the instrument itself.

In each of the example methods described by means of FIGS. 7A-7D, when the marker comprises a medical device identification code, the method may further comprise the detection device reading the identification code (a step not shown in the Figures).

The described methods may further comprise a prior step of marking the medical device, by affixing said marker integrally to said medical device, when the marker is an external element to the device (step not shown in the Figures).

Although described through a number of embodiments, the methods for detecting a medical instrument and the disinfection devices configured for implementing these methods comprise different variants, modifications and improvements that will be obvious to the person skilled in the art, it being understood that these different variants, modifications and improvements are within the scope of the invention as defined by the following claims.

Thus, for example, medical instruments without connection cables can also be disinfected by means of the disinfection methods and systems described in the present description, by creating a receptacle transparent to UV radiation for example, or by attaching to the medical instrument an element in the form of a “cap” which is then suspended.

In addition, instead of UV radiation such as that obtained from a UV radiation source such as UV-C, embodiments may use a “flash” type energy source emitting extremely high intensity radiation, including electron beam radiation, gamma radiation, x-radiation, or plasma gas radiation. For example, the flash-type energy source may include a light emitting diode (LED).

1. A disinfection system configured to implement a disinfection cycle of a medical instrument by UV radiation, the disinfection system comprising:
a disinfection chamber configured to receive at least one portion of the medical instrument;
a suspension means for suspending said at least one portion of the medical instrument in said disinfection chamber;
a detection device configured to detect a marker integral with said medical instrument, in a predetermined detection area of the disinfection system; and
a controller configured to generate a non-detection signal in case of non-detection of said marker in said predetermined detection area.

2. The disinfection system according to claim 1, wherein the detection device comprises an optical detection device.

3. The disinfection system according to claim 2, wherein the detection device further comprises a source for emitting light radiation configured to illuminate said predetermined detection area.

4. The disinfection system according to claim 2, wherein the detection device comprises a barcode reader.

5. The disinfection system according to claim 1, wherein the detection device comprises radio frequency acquisition means.

6. The disinfection system according to claim 1, comprising at least one source of UV radiation.

7. A detection kit for a medical instrument to be disinfected in a disinfection system according to claim 1, said medical instrument comprising a head and a connection cable of the head, the detection kit comprising:
a marker configured to be integrally affixed to a portion of the connection cable adjacent the head for detection by said detection device; and
a protective ring of said marker, said protective ring further interacting with said suspension means for suspending the connection cable.

8. The detection kit of claim 7, wherein the marker comprises an identification code of said medical instrument.

9. A disinfection kit for a medical instrument comprising the disinfection system according to claim 2, said medical instrument comprising a head and a connection cable of the head, the detection kit comprising:
a marker configured to be integrally affixed to a portion of the connection cable adjacent the head for detection by said detection device; and
a protective ring of said marker, said protective ring further interacting with said suspension means for suspending the connection cable.

10. A method for detecting a medical instrument in a disinfection system according to claim 1, the method comprising the following steps:
detecting said marker integral with said medical instrument in said predetermined detection area of the disinfection system, by means of said detection device; and
in case of non-detection of said marker in said predetermined detection area of the disinfection system, generating a non-detection signal.

11. The detection method according to claim 10, wherein generation of the non-detection signal results in issuing an alert to one or more users and/or stopping a disinfection cycle in progress and/or prohibiting the launch of a cycle.

12. The detection method according to claim 10, wherein the marker comprises an identification code of the medical instrument and the method further comprises:
reading the identification code using the detection device.

13. The detection method according to claim 10, comprising a prior step of marking the medical instrument, by affixing the marker integrally to said medical instrument.

14. The detection method according to claim 10, wherein the detection is optical detection or radio detection.

15. The detection method according to claim 10, further comprising detecting, in said detection area, a position of the marker relative to a reference position.

16. The detection method according to claim 15, further comprising one or more actions, based on said position of the marker in the detection area, comprising: issuing a positioning error alert to one or more user(s) and/or reconfiguring a disinfection cycle to take account of said position and/or stopping the disinfection cycle in progress and/or prohibiting the launch of a disinfection cycle.
